(19) United States
(12) Patent Application Publication
(10) Pub. No.: US 2024/0225910 A1
(43) Pub. Date: Jul. 11, 2024

(54) ABSORBENT ARTICLE

(71) Applicant: Luxwear LLC, Pittsburgh, PA (US)

(72) Inventor: Lindsay Paige Stuart, Pittsburgh, PA (US)

(73) Assignee: Luxwear LLC, Pittsburgh, PA (US)

(21) Appl. No.: 18/094,721

(22) Filed: Jan. 9, 2023

(51) Int. Cl. A61F 13/14

(52) U.S. Cl. CPC A61F 13/14; A61F 2220/0083; A61F 2250/0076

(57) ABSTRACT

An absorbent article for positioning within a garment, the absorbent article having an absorbent inner layer, a moisture proof outer layer, and a binding edge connecting peripheral edges of the inner and outer layers together, wherein the absorbent article is shaped and sized to cover at least a portion of a breast of person with the inner layer facing the breast. Also provided is an article of clothing having a cutout, wherein the cutout is configured to removably accept and support the absorbent article, wherein an inner layer of the absorbent article contacts the wearer's skin and wherein an outer layer of the absorbent article removably attaches to the article of clothing.

### CROSS-REFERENCE TO RELATED APPLICATION

This application claims priority to and the benefit of U.S. Provisional Patent Application No. 63/297,775, filed on Jan. 9, 2022, entitled ABSORBENT ARTICLE, which is incorporated herein by reference in its entirety.

### FIELD OF THE INVENTION

This disclosure generally relates to the field of absorbent articles and articles of clothing comprising the same.

### BACKGROUND

During pregnancy, human breasts enlarge under the stimulation of hormones generated by the body. Once the placenta is delivered, milk may be secreted from the nipples of the breast. Milk secretion from the breast is initiated by prolactin and is maintained by nipple-sucking stimulation. During the period of time breast feeding is maintained, the breasts and nipples are highly sensitive and may secrete milk even when suckling is not occurring. Conventional absorbent articles for preventing breast milk leakage from contacting and seeping into and through clothing and bed linens are currently commercially available. Generally, these types of devices fall into two broad categories: nursing or breast pads and nursing or protective brassieres. These two categories each comprise two general sub categories, reusable pads and disposable pads, and unitary brassieres and brassieres having replaceable absorbent pads. The conventional absorbent articles are external or separate from a wearer's garment top or brassiere. The nursing pads may be placed between the breast and the garment top or brassiere.

Conventional absorbent articles may suffer from one or more of the following limitations. Conventional absorbent articles may lack sufficient absorbent material and/or absorbent characteristics and thus fail to prevent the breast milk from leaking through the pad onto the garment top, brassiere, or bed linen. As a result, the wearer may experience an embarrassing leak and possibly damaging stains on the garment top, brassiere, or bed linen. In addition, conventional absorbent articles may leak when the pads do not stay in place. Conventional absorbent articles may also be inconvenient and/or time consuming since the pad must be removed for access during nursing. Conventional absorbent articles that are separate from the garment or brassiere may become lost or otherwise misplaced. Once nursing is complete and when the absorbent article is not misplaced or otherwise lost, conventional absorbent articles may need to be repositioned between the breast and the garment top or brassiere, which may be inconvenient and/or time consuming. Certain materials used in conventional absorbent articles may cause discomfort and/or pain when made of materials that stick to nipples, causing skin irritation. Many conventional nursing pads may be also visible through the garment top.

Accordingly, more efficient and/or cost-effecting absorbent articles suitable for absorbing lacteal fluid that is comfortable to the human breast may be desirable.

### SUMMARY

The present disclosure is directed to more efficient and/or cost-effecting absorbent articles suitable for absorbing lacteal fluid that is comfortable to the human breast.

The absorbent article may comprise an absorbent inner layer, a moisture proof outer layer, and a binding edge connecting peripheral edges of the inner and outer layers together, wherein the absorbent article is shaped and sized to cover at least a portion of a breast of a person with the inner layer facing the breast. The absorbent article may be positioned within a garment. The absorbent article may comprise an intermediate layer positioned between the inner layer and the outer layer that is joined along a peripheral edge.

The present disclosure is directed to an article of clothing. The article of clothing may comprise a cutout, wherein the cutout is configured to removably accept and support the absorbent article of the present disclosure, wherein an inner layer of the absorbent article contacts the wearer's skin and wherein an outer layer of the absorbent article removably attaches to the article of clothing.

The present disclosure is directed to an article of clothing comprising absorbent articles suitable for absorbing lacteal fluid that is comfortable to the human breast.

The present disclosure is directed to methods of using absorbent articles suitable for absorbing lacteal fluid that is comfortable to the human breast.

The present disclosure is directed to methods of manufacturing absorbent articles suitable for absorbing lacteal fluid that is comfortable to the human breast.

### DESCRIPTION OF THE DRAWINGS

The present invention described herein may be better understood by reference to the accompanying drawing sheets, in which:

FIG. 1 includes an illustration of an absorbent article according to the present invention.

FIG. 2 includes an illustration of a garment configured to support an absorbent article according to the present invention.

FIG. 3 includes an illustration of an absorbent article according to the present invention removably coupled to a garment.

FIG. 4 includes an illustration of a cross section view of an absorbent article of FIG. 1.

### DETAILED DESCRIPTION

This disclosure generally describes absorbent articles as well as methods of making and using the same. It is understood, however, that this disclosure also embraces numerous alternative features, aspects, and advantages that may be accomplished by combining any of the various features, aspects, and/or advantages described herein in any combination or sub-combination that one of ordinary skill in the art may find useful. Such combinations or sub-combinations are intended to be included within the scope of this disclosure. As such, the claims may be amended to recite any features, aspects, and advantages expressly or inherently described in, or otherwise expressly or inherently supported by this disclosure. Further, any features, aspects, and advantages that may be present in the prior art may be affirmatively disclaimed. Accordingly, this disclosure may comprise, consist of, consist essentially of or be characterized by one or more of the features, aspects, and advantages described herein.

All numerical quantities stated herein may be approximate, unless stated otherwise. Accordingly, the term “about” may be inferred when not expressly stated. The numerical quantities disclosed herein may be to be understood as not being strictly limited to the exact numerical values recited. Instead, unless stated otherwise, each numerical value stated herein is intended to mean both the recited value and a functionally equivalent range surrounding that value. At the very least, and not as an attempt to limit the application of the doctrine of equivalents to the scope of the claims, each numerical value should at least be construed in light of the number of reported significant digits and by applying ordinary rounding processes. Typical exemplary degrees of error may be within 20%, 10%, or 5% of a given value or range of values. Alternatively, the term “about” refers to values within an order of magnitude, potentially within 5-fold or 2-fold of a given value. Notwithstanding the approximations of numerical quantities stated herein, the numerical quantities described in specific examples of actual measured values may be reported as precisely as possible. Any numerical values, however, inherently contain certain errors necessarily resulting from the standard deviation found in their respective testing measurements.

All numerical ranges stated herein include all sub-ranges subsumed therein. For example, a range of “1 to 10” or “1-10” is intended to include all sub-ranges between and including the recited minimum value of 1 and the recited maximum value of 10 because the disclosed numerical ranges may be continuous and include every value between the minimum and maximum values. Any maximum numerical limitation recited herein is intended to include all lower numerical limitations. Any minimum numerical limitation recited herein is intended to include all higher numerical limitations.

Unless otherwise defined, all terms (including technical and scientific terms) used herein have the same meaning as commonly understood by one of ordinary skill in the art to which this disclosure belongs. As such, terms, such as those defined by commonly used dictionaries, should be interpreted as having a meaning that is consistent with their meaning in a context of a relevant art and should not be interpreted in an idealized or overly formal sense unless expressly so defined herein.

As generally used herein, the terms “inner” and “outer” refer respectively to the location of an element that is intended to be placed against or toward the body of a wearer when an absorbent article is worn and the location of an element that is intended to be placed against or toward any clothing that is worn over the absorbent article. Synonyms for “inner” and “outer” include, respectively, “interior” and “exterior”, as well as “inside” and “outside”. As used herein, the terms “inner” and “outer” are used interchangeably with “interior” and “exterior”, respectively. Also, when the absorbent article is oriented such that its interior faces upward, e.g., when it is laid out in preparation for setting the wearer on top of it, synonyms include “upper” and “lower” and “top” and “bottom”, respectively.

The term “lateral” or “transverse” refers to a direction running at a 90 degree angle to the longitudinal direction and includes directions within ±45 degree of the lateral direction. The term “longitudinal” refers to a direction running parallel to the maximum linear dimension of the article and includes directions within ±45 degree of the longitudinal direction. “Horizontal” refers to a plane extending from left to right and aligned with the horizon, and “vertical” refers to a plane that is angled at 90 degrees to the horizontal.

The term “disposed” refers to an element(s) formed (joined and positioned) in a particular place or position as a unitary structure with other elements or as a separate element joined to another element.

The term “joined” as used herein refers to configurations whereby an element is directly secured to another element, and configurations whereby an element is indirectly secured to another element by attaching.

The terms “releasably supported”, “releasably attached”, “releasably engaged”, and variations thereof refer to two elements being connected or connectable such that the elements tend to remain connected absent a separation force applied to one or both of the elements, and the elements being capable of separation without substantial permanent deformation or rupture. The required separation force is typically beyond that encountered while wearing the absorbent garment.

Note that various terminology used herein may imply direct or indirect, full or partial, temporary or permanent, action or inaction. For example, when an element is referred to as being “on,” “connected”, or “coupled” to another element, then the element may be directly on, connected or coupled to the other element or intervening elements may be present, including indirect or direct variants. In contrast, when an element is referred to as being “directly connected” or “directly coupled” to another element, there may be no intervening elements present.

Furthermore, when this disclosure states that something is “based on” something else, then such statement refers to a basis which may be based on one or more other things as well. In other words, unless expressly indicated otherwise, as used herein “based on” inclusively means “based at least in part on” or “based at least partially on.”

As used herein, the term “and/or” includes any and all combinations of one or more of the associated listed items. Likewise, as used in the following detailed description, the term “or” is intended to mean an inclusive “or” rather than an exclusive “or.” That is, unless specified otherwise, or clear from context, “X employs A or B” is intended to mean nay of the natural inclusive permutations. Thus, if X employs A; X employs B; or X employs both A and B, then “X employs A or B” is satisfied under any of the foregoing instances.

The terminology used herein is for the purpose of describing particular examples only and is not intended to be limiting. As used herein, the singular forms “a”, “an”, and “the” may be intended to include the plural forms as well, unless the context clearly dictates otherwise. As example, “a” layer may comprise one or more layers, and the like.

The terms “comprises”, “comprising”, “including”, “having”, and “characterized by”, may be inclusive and therefore specify the presence of stated features, elements, compositions, steps, integers, operations, and/or components, but do not preclude the presence or addition of one or more other features, integers, steps, operations, elements, components, and/or groups thereof. Although these open-ended terms may be to be understood as a non-restrictive term used to describe and claim various aspects set forth herein, in certain aspects, the term may alternatively be understood to instead be a more limiting and restrictive term, such as “consisting of” or “consisting essentially of.” Thus, for any given embodiment reciting compositions, materials, components, elements, features, integers, operations, and/or process steps, described herein also specifically includes embodiments consisting of, or consisting essentially of, such recited compositions, materials, components, elements, features, integers, operations, and/or process steps. In the case of “consisting of”, the alternative embodiment excludes any additional compositions, materials, components, elements, features, integers, operations, and/or process steps, while in the case of “consisting essentially of”, any additional compositions, materials, components, elements, features, integers, operations, and/or process steps that materially affect the basic and novel characteristics may be excluded from such an embodiment, but any compositions, materials, components, elements, features, integers, operations, and/or process steps that do not materially affect the basic and novel characteristics may be included in the embodiment.

Any method steps, processes, and operations described herein may not be construed as necessarily requiring their performance in the particular order discussed or illustrated, unless specifically identified as an order of performance. It is also to certain example embodiments may be combined in or with various other example embodiments in any permutational or combinatory manner. Different aspects or elements of example embodiments, as disclosed herein, may be combined in a similar manner. The term “combination”, “combinatory,” or “combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included may be combinations that contain repeats of one or more item or term, such as BB, AAA, AB, BBC, AAABCCCC, CBBAAA, CABABB, and so forth. The skilled artisan will understand that typically there is no limit on the number of items or terms in any combination, unless otherwise apparent from the context.

Words such as “then,” “next,” etc. are not intended to limit the order of the steps; these words may be simply used to guide the reader through the description of the methods. In addition, the order of the operations may be re-arranged.

In the following description, certain details are set forth in order to provide a better understanding of various features, aspects, and advantages of the presently disclosed invention. However, one skilled in the art will understand that these features, aspects, and advantages may be practiced without these details. In other instances, well-known structures, methods, and/or processes may not be shown or described in detail to avoid unnecessarily obscuring descriptions of other details of the invention.

Features or functionality described with respect to certain example embodiments may be combined and sub-combined in and/or with various other example embodiments. Also, different aspects and/or elements of example embodiments, as disclosed herein, may be combined and sub-combined in a similar manner as well. Further, some example embodiments, whether individually and/or collectively, may be components of a larger system, wherein other procedures may take precedence over and/or otherwise modify their application. Additionally, a number of steps may be required before, after, and/or concurrently with example embodiments, as disclosed herein. Note that any and/or all methods and/or processes, at least as disclosed herein, may be at least partially performed via at least one entity or actor in any manner.

The present invention is directed to an absorbent article suitable for absorbing lacteal fluid that is comfortable to the human breast.

The present invention is directed to methods of using absorbent articles suitable for absorbing lacteal fluid that is comfortable to the human breast.

The present invention is directed to methods of manufacturing absorbent articles suitable for absorbing lacteal fluid that is comfortable to the human breast.

The present invention is directed to a breast pad suitable for absorbing lacteal fluid that is comfortable to the human breast.

The present invention is directed to an article of clothing comprising absorbent articles suitable for absorbing lacteal fluid that is comfortable to the human breast. The absorbent article may minimize the soiling of clothing worn by the user.

The present invention is directed to an article of clothing comprising a breast pad suitable for absorbing lacteal fluid that is comfortable to the human breast. The breast pad may minimize the soiling of clothing worn by the user.

The absorbent article may comprise at least one of absorbent underwear clothing in the nature of breast-nursing pads; inserts specifically adapted for nursing bras, namely, nursing pads; clothing shields, namely, pads that may be affixed directly to the inside of clothing; clothing shields, namely, pads applied to the inside of tops; inserts specifically adapted for nursing bras for primarily non-medical purposes; readymade linings being parts of clothing; bottoms as clothing; postpartum clothing, namely, tops, shirts, underwear, dresses, robes, bottoms; ladies' underwear; bras; nursing bras; maternity bras, maternity tops; and slippers and socks.

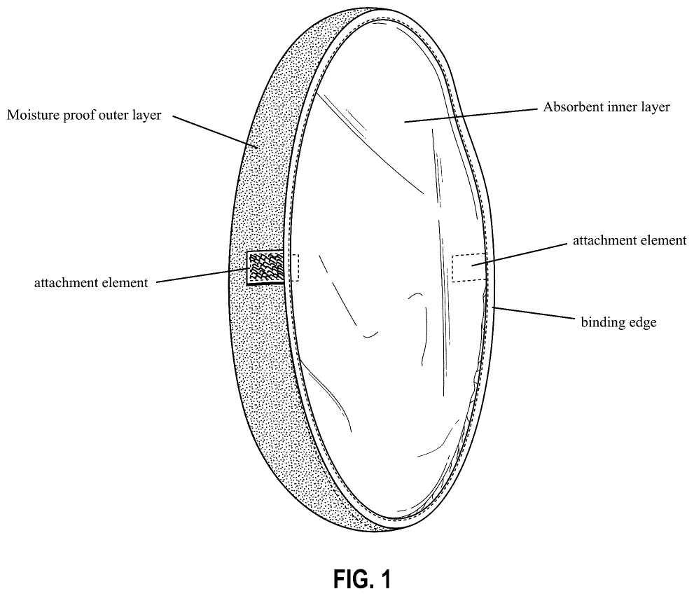

Referring to FIG. 1, the absorbent article may be connected to or positioned within a garment or article of clothing. The absorbent article may comprise an absorbent inner layer, a moisture proof outer layer, and a binding edge connecting the peripheral edges of the inner and outer layers together. The absorbent article may comprise an intermediate layer having a peripheral edge positioned between the inner layer and the outer layer. The intermediate layer may be joined to the inner and outer layers, such as along the peripheral edge. A binding edge may connect the peripheral edges of inner, intermediate, and outer layers together.

The inner layer may contact the wearer's skin and may comprise fabric, which may be the same or different from the outer layer. The inner layer may be characterized by antimicrobial and/or moisture-wicking properties. The inner layer fabric may include, but is not limited to, bamboo blend fabric, polyester, polypropylene, Merino wool, wool, nylon, MicroModal, rayon, polyester-vinyl composites, vinyl, acrylics, any combination thereof, or any other textile or composition that provides moisture-wicking properties and/or protection against bacteria, mold, mildew, and other hazardous microbes. The inner layer may comprise one of a concave surface and a convex surface. The concave surface may be positioned proximate to the wearer's body. The shape of the surface of the inner layer may provide one or more advantages relative to a flat surface, such as, better leak protection, more support, and a more seamless look.

The absorbent article may comprise an outer layer opposed to the inner layer. The outer layer may comprise a fabric the same or different from the inner layer. The outer layer may be characterized by leak protection and/or waterproof properties. The outer layer fabric may include, but is not limited to, polyester, polypropylene, Merino wool, wool, nylon, MicroModal, rayon, polyester-vinyl composites, vinyl, acrylics, laminated cotton, cotton, polyurethane laminate, thermoplastic polyurethane, Zorb® 4D Polyester Dimple Waterproof CORE PUL Soaker Silver Fabric, any combination thereof, or any other textile or composition that provides leak protection and/or waterproof properties. The outer layer may comprise Zorb® 4D Polyester Dimple Waterproof CORE PUL Soaker Silver Fabric.

The absorbent article may comprise an intermediate layer positioned between the inner layer and the outer layer. The intermediate layer may be characterized by absorbent and fast-drying properties. The term “fast-drying”, as used herein, is used interchangeably with the terms “quick-dry” and “quick-drying.” The term “fast-drying” refers to the ability of fabric to dry completely within 24 hours upon becoming saturated or in contact with a fluid or liquid. The intermediate layer may comprise microfiber terry fabric. The intermediate layer fabric may include, but is not limited to, cotton, wool, merino wool, linen, bamboo, nylon, polyester, rayon, silk, microfiber terry fabric, any combination thereof, or any other textile or composition that provides absorbent and/or fast-drying properties.

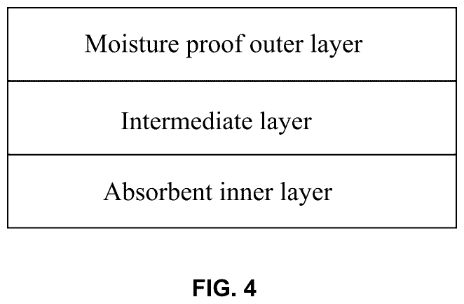

Referring to FIG. 4, the outer layer may be disposed on the intermediate layer and the intermediate layer may be disposed on the inner layer. In some embodiments, the absorbent article may lack an intermediate layer such that the outer layer is disposed on the inner layer.

The outer layer may be joined to the intermediate layer and the intermediate layer may be joined to the inner layer. In some embodiments, the absorbent article may lack an intermediate layer such that the inner layer is joined to the outer layer.

The binding edge connecting the peripheral edges of the inner and outer layers, or the inner, intermediate, and outer layers may comprise stitching, an adhesive or sealant, a fabric binding, or any combination thereof.

The absorbent article may be attached to an article of clothing or garment by fastening, adhering, bonding, or any other method suitable for connecting or uniting the absorbent article to the garment, such as, adhesive bonding, pressure bonding, thermal bonding, ultrasonic bonding, mechanical fastening, etc. Such attachment methods may be used to attach the absorbent article and garment together over a particular area either continuously or intermittently.

The absorbent article may comprise an attachment system comprising at least one attachment element. The attachment system may be any suitable attachment system capable of providing removeable attachment of the absorbent article to an article of clothing. For example, the attachment system may be a hook and loop system, such as VELCRO™, a low-strength adhesive, hook and eye fasteners, or the like.

For example, the absorbent article may comprise a loop fastener, such as VELCRO™ attached to the outer layer of the absorbent article. The article of clothing may comprise a hook fastener such as VELCRO™ to attach the loop fastener such as VELCRO™ located on the outer layer of the absorbent article. The outer layer of the absorbent article may comprise a hook fastener such as VELCRO™ to removably couple the absorbent article to the article of clothing, wherein the article of clothing comprises a corresponding hook and loop closure element (e.g., loop closure).

The absorbent article may be releasably supported by the garment such that the absorbent article is capable of separation without substantial permanent deformation, damage, or rupture. The absorbent article may then be attached to another article or clothing or garment.

Thus, the absorbent article may be refastenable to the garment or a different garment. The term “refastenable” refers to the characteristic of two elements being capable of releasable attachment, separation, and subsequent releasable attachment, separation, and subsequent releasable reattachment without substantial permanent deformation, damage, or rupture.

The absorbent article may be disposable or non-disposable. The term “disposable” refers to absorbent articles of the present disclosure that generally are not intended to be laundered or otherwise restored or reused as absorbent articles, i.e., they are intended to be discarded after single use and, preferably, to be recycled, composted, or otherwise disposed of in an environmentally compatible manner.

The absorbent article may comprise clothing shields, namely, pads applied to the inside of articles of clothing or garments to protect clothing from the various fluids. The absorbent article may be placed against or in proximity to the body of the wearer to absorb and contain the various fluids, including, but not limited to, breast milk, blood, or other bodily fluids. The clothing shields may be applied inside the tops of articles of clothing.

The absorbent article may be shaped and sized to cover at least a portion of a breast of a person. The size and shape of the absorbent article may correspond to bra cup sizes, including, but not limited to, AA, A, B, C, D, DD, DDD, E, F, G, H, or the like. The absorbent article may be shaped and sized to conform to atypical breasts. Atypical breasts include but are not limited to, asymmetrical, athletic, bell shape, close set, conical, east west, relaxed, slender, teardrop, and sideset breasts.

The absorbent article may have a diameter from 5-25 cm, such as 9.7-22.4 cm, 10.6-21.6 cm, 11.4-20.7 cm, 12.3-20.7 cm, 13.1-19.9 cm, 14-19 cm, 14.8-18.2 cm, 15.7-17.4 cm, 7-9 cm, 7.5-8.5 cm, and 8 cm. The diameter of the inner layer and the diameter of the outer layer may be the same or different. The inner layer may have a diameter from 5-25 cm, such as 9.7-22.4 cm, 10.6-21.6 cm, 11.4-20.7 cm, 12.3-20.7 cm, 13.1-19.9 cm, 14-19 cm, 14.8-18.2 cm, 15.7-17.4 cm, 7-9 cm, 7.5-8.5 cm, 5 cm, 6 cm, 7 cm, and 8 cm. The outer layer may have a diameter from 5-25 cm, such as 9.7-22.4 cm, 10.6-21.6 cm, 11.4-20.7 cm, 12.3-20.7 cm, 13.1-19.9 cm, 14-19 cm, 14.8-18.2 cm, 15.7-17.4 cm, 7-9 cm, 7.5-8.5 cm, 5 cm, 6 cm, 7 cm, and 8 cm. The diameter of the adhesive material may be equal to or less than the diameter of the inner layer and/or outer layer. The adhesive material may have a diameter from 1-20 cm, such as 1-8 cm, 9.7-22.4 cm, 10.6-21.6 cm, 11.4-20.7 cm, 12.3-20.7 cm, 13.1-19.9 cm, 14-19 cm, 14.8-18.2 cm, 15.7-17.4 cm, 7-9 cm, 7.5-8.5 cm, 5 cm, 6 cm, 7 cm, and 8 cm. The absorbent article may have a width from 0.1-10 cm, such as 1-10 cm, 0.5-2 cm, 0.75-1 cm, and 1 cm.

The absorbent article may be configured to have a concave shape on an inner surface, wherein the inner surface is positionable proximate to a wearer's body. The absorbent article may be configured to be contoured or not flat. The absorbent article, when attached to an article of clothing or garment, may conform to the concave slope across the upper pole of a breast and the convex shape across the lower pole of a breast. The absorbent article may be configured to lay flat across the chest or other body part of a wearer.

The absorbent article may be adapted for and may be inserted in nursing bras. The present disclosure also includes nursing bras comprising cutouts or readymade linings, wherein at least one absorbent article may be inserted into the article of clothing or garment.

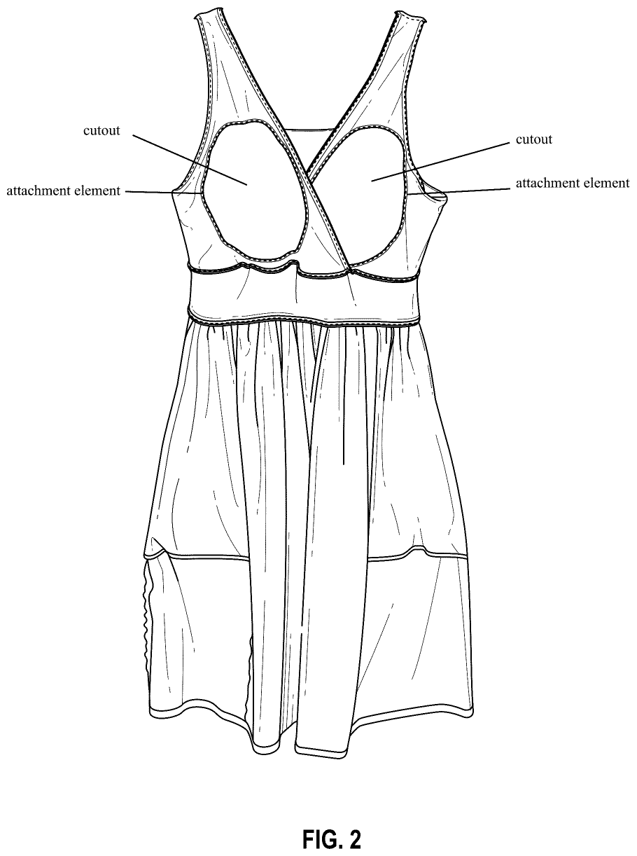

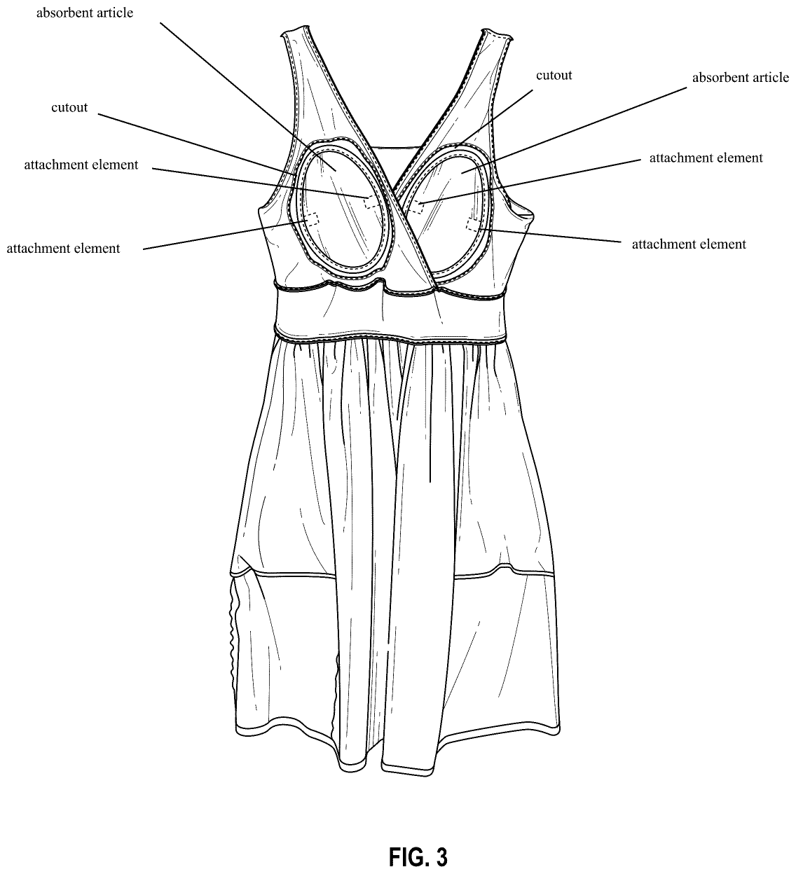

Referring to FIGS. 2 and 3, the present disclosure also provides for an article of clothing comprising a cutout, wherein the cutout is configured to removably accept and support the absorbent article of the present disclosure wherein the inner layer of the absorbent article contacts the wearer's skin and the outer layer of the absorbent article removably attaches to the article of clothing. The cutout may be shaped and sized to cover at least a portion of a breast of a person. The cutout may comprise at least one hook and loop closure element configured to receive and secure the absorbent article via corresponding hook and loop closure elements positioned on the outer layer of the absorbent article.

The cutout may be configured to allow the absorbent article to more easily attach to the article of clothing or garment. The cutout may be positioned anywhere within a predetermined area where each wearer feels comfortable. The breast pad may not be visible through the garment top.

The present invention is directed to a breast pad having an absorbent front side which faces the breast and a moisture resistant/proof back side that faces the garment. The breast pad may comprise an adhesive configured to adhere the breast pad to the breast. The breast pad may comprise a backing to maintain the adhesive material's ability to attach the breast pad to the breast and/or garment, as applicable, when not in use. The backing may comprise a shape corresponding to the shape of the adhesive material.

The present invention is directed to a method of using the breast pad. The method may comprise providing a breast pad having an adhesive material and a backing, removing the backing to expose the adhesive material, and applying the breast pad to the breast by adhering the adhesive material to the breast and/or applying the breast pad to the garment by adhering the adhesive material to the garment.

The present invention is directed to a method of making a breast pad. The method may comprise applying an adhesive material to an absorbent front side of the breast pad which faces the breast and/or applying an adhesive material to a moisture resistant/proof back side of the breast pad that faces the garment. The method may comprise applying a backing to the adhesive material to protect the adhesive before use. The method may include layering at least one additional layer of intermediate between the front side and back side of the breast pad. The method may comprise applying a barrier layer that is a moisture resistant/proof to the back side of the breast pad that faces the garment. The method may comprise compressing the front side, at least one additional layer, and back side together to form the breast pad.

The present invention is directed to a plurality of absorbent articles suitable for absorbing lacteal fluid that is comfortable to the human breast that may be stored and transported in a container. The container may include a bag, a box, or a tube. The breast pad may be individually wrapped in the container. The container may be configured to store and transport 2-100 breast pads, such as 30 or 100.

The following aspects are disclosed in this application:
Aspect 1: An absorbent article for positioning within a garment, the article comprising: an absorbent inner layer; a moisture proof outer layer; and a binding edge connecting peripheral edges of the inner and outer layers together, wherein the absorbent article is a breast pad.
Aspect 2: The absorbent article of aspect 1, further comprising: an intermediate layer having a peripheral edge, wherein the intermediate layer is positioned between the inner layer and the outer layer and is joined thereto along the peripheral edge, and wherein the intermediate layer comprises absorbent and fast-drying fabric.
Aspect 3: The absorbent article of any of the foregoing aspects, wherein the absorbent article is configured to have a concave shape on an inner surface, wherein the inner surface is positionable proximate to a wearer's body.
Aspect 4: The absorbent article of any of the foregoing aspects, wherein the inner layer comprises an antimicrobial fabric.
Aspect 5: The absorbent article of any of the foregoing aspects, wherein the outer layer comprises a waterproof fabric.
Aspect 6: The absorbent article of any of the foregoing aspects, wherein the absorbent article comprises at least one attachment element configured to provide removeable attachment of the absorbent article to an article of clothing.
Aspect 7: The absorbent article of any of the foregoing aspects, wherein the at least one attachment element comprises hook and loop closure.
Aspect 8: The absorbent article of any of the foregoing aspects, wherein the absorbent article is shaped and sized to cover at least a portion of a breast of a person.
Aspect 9: An article of clothing comprising, a cutout, wherein the cutout is configured to removably accept and support the absorbent article of any of the foregoing aspects, wherein an inner layer of the absorbent article contacts the wearer's skin and wherein an outer layer of the absorbent article removably attaches to the article of clothing.
Aspect 10: The article of clothing of Aspect 9, comprising at least one hook and loop closure element configured to receive and secure the absorbent article via corresponding hook and loop closure elements positioned on the outer layer thereof.
Aspect 11: The article of clothing of Aspect 9 or 10, wherein the cutout is shaped and sized to cover at least a portion of a breast of a person.

All documents cited herein may be incorporated herein by reference, but only to the extent that the incorporated material does not conflict with existing definitions, statements, or other documents set forth herein. To the extent that any meaning or definition of a term in this document conflicts with any meaning or definition of the same term in a document incorporated by reference, the meaning or definition assigned to that term in this document shall govern. The citation of any document is not to be construed as an admission that it is prior art with respect to this application.

While particular embodiments have been illustrated and described, it would be obvious to those skilled in the art that various other changes and modifications may be made without departing from the spirit and scope of the invention. Those skilled in the art will recognize or be able to ascertain using no more than routine experimentation, numerous equivalents to the specific apparatuses and methods described herein, including alternatives, variants, additions, deletions, modifications and substitutions. This application including the appended claims is therefore intended to cover all such changes and modifications that may be within the scope of this application.

What is claimed is:

1. An absorbent article for positioning within a garment, the article comprising:
an absorbent inner layer;
a moisture proof outer layer; and
a binding edge connecting peripheral edges of the inner and outer layers together,
wherein the absorbent article is shaped and sized to cover at least a portion of a breast of a person with the inner layer facing the breast;
wherein the absorbent article is a breast pad.

2. The absorbent article of claim 1, further comprising:
an intermediate layer having a peripheral edge, wherein the intermediate layer is positioned between the inner layer and the outer layer and is joined thereto along the peripheral edge, and wherein the intermediate layer comprises absorbent and fast-drying fabric.

3. The absorbent article of claim 1, wherein the absorbent article is configured to have a concave shape on an inner surface, wherein the inner surface is positionable proximate to a wearer's body.

4. The absorbent article of claim 1, wherein the inner layer comprises an antimicrobial fabric.

5. The absorbent article of claim 1, wherein the outer layer comprises a waterproof fabric.

6. The absorbent article of claim 1, wherein the absorbent article comprises at least one attachment element configured to provide removeable attachment of the absorbent article to an article of clothing.

7. The absorbent article of claim 6, wherein the at least one attachment element comprises hook and loop closure.

8. An article of clothing comprising,
a cutout, wherein the cutout is configured to removably accept and support the absorbent article of claim 1, wherein an inner layer of the absorbent article contacts the wearer's skin and wherein an outer layer of the absorbent article removably attaches to the article of clothing.

9. The article of clothing of claim 8, comprising at least one hook and loop closure element configured to receive and secure the absorbent article via corresponding hook and loop closure elements positioned on the outer layer thereof.

10. The article of clothing of claim 8, wherein the cutout is shaped and sized to cover at least a portion of a breast of a person.
